(19) United States
(12) Patent Application Publication
(10) Pub. No.: US 2004/0029974 A1
(43) Pub. Date: Feb. 12, 2004

(54) Packaging articles having insect-combating characteristics, packaged goods comprising such packaging articles, and methods of making the same

(76) Inventors: Alan Brandt, Chapel Hill, NC (US); John Bennett, Chapel Hill, NC (US)

Correspondence Address: INTELLECTUAL PROPERTY / TECHNOLOGY LAW, PO BOX 14329, RESEARCH TRIANGLE PARK, NC, 27709, US

(21) Appl. No.: 10/609,356

(22) Filed: Jun. 27, 2003

Related U.S. Application Data
(60) Provisional application No. 60/394,013, filed on Jul. 3, 2002.

(51) Int. Cl. A61K 31/12

(52) U.S. Cl. 514/675

(57) ABSTRACT

Packaging and packaged products susceptible to insect attack are afforded protective character against such attack by incorporation in the packaging an insect-combating amount of 2-undecanone or 2-tridecanone.

### CROSS-REFERENCE TO RELATED APPLICATION

The priority of U.S. Provisional Patent Application No. 60/394,013 filed Jul. 3, 2002 in the names of Alan Brandt and John Bennett entitled “PACKAGING ARTICLES HAVING INSECT-COMBATING CHARACTERISTICS, PACKAGED GOODS COMPRISING SUCH PACKAGING ARTICLES, AND METHODS OF MAKING THE SAME” is hereby claimed under the provisions of 35 USC 119.

### BACKGROUND OF THE INVENTION

1. Field of the Invention

The present invention relates generally to packaging articles having an inherent insect-combating characteristic, e.g., insect repellency or insecticidal character, and to packaged goods including such packaging articles, as well as to a method of making such packaging articles.

2. Description of the Prior Art

In the field of packaging, involving packaging structures and materials for containment, storage, shipping and/or protection of packaged goods, there are a number of applications in which the packaging article or material of construction, as well as the contained object or material, is susceptible to insect attack. Examples include packaging materials such as cellulosic, fabric, polymeric, etc. materials that are intrinsically subject to such attack, as well as contained goods that are susceptible to insect attack such as human and/or veterinary food/nutrition products, cosmetics, pharmaceuticals, and other organic and inorganic materials.

Among insects that are potentially problematic in such respect are ticks, fleas, aphids, cockroaches, flies (horse flies, deer flies, black flies, etc.), gnats, no-see ums, chiggers, thrips, mosquitoes, beetles (e.g., Colorado potato beetle), etc., including larvae as well as adult insects.

It would be an advance in the art to provide packaging components and materials having an inherently insect-combating characteristic, e.g., insect-repellency and/or insecticidal character that is specific for insects such as those identified above.

### SUMMARY OF THE INVENTION

The present invention relates in one aspect to a packaging article, comprising a package structure, component or material of construction, containing an insect-combating amount of 2-undecanone or 2-tridecanone.

In another aspect, the invention relates to a packaged product comprising a package including a package structure, component or material of construction, containing an insect-combating amount of 2-undecanone or 2-tridecanone, and a contained product in said package.

A further aspect of the invention relates to a method of combating insects to which a packaging is susceptible, comprising fabricating said packaging with an incorporated insect-combating amount of 2-undecanone or 2-tridecanone.

Yet another aspect of the invention relates to a method of combating insects to which a packaging and/or its contents is susceptible, comprising fabricating said packaging with an incorporated insect-combating amount of 2-undecanone or 2-tridecanone.

Other aspects, features and embodiments of the present invention will be more fully apparent from the ensuing disclosure and appended claims.

### BRIEF DESCRIPTION OF THE DRAWING

FIG. 1 is a schematic representation of a packaged product including a package at least some of whose components contain an insect-combating amount of 2-undecanone or 2-tridecanone.

### DETAILED DESCRIPTION OF THE INVENTION, AND PREFERRED EMBODIMENTS THEREOF

The disclosures of the following patents and patent applications are hereby incorporated herein by reference in their respective entireties: U.S. patent application Ser. No. 09/808,499 filed Mar. 14, 2001 in the name of R. Michael Roe, now allowed; U.S. Pat. No. 5,788,975; and U.S. Pat. No. 6,395,290.

The present invention is based on the discovery that insect-combating agents, viz., insect repellants and insecticides, are usefully incorporated in packaging as intrinsic ingredients of the packaging structure, materials and/or components, to provide a protective function with respect to the packaging per se and/or the contained product of or in the packaging. The invention thus achieves a substantial advance over the prior art approach of localized application of insect-combating agents to environments in which the packaged product is disposed, such prior art approach being extremely wasteful of the insect-combating agent, which has a tendency to be overapplied, or very unevenly applied, and whose insect-combating activity is confined to such locus, so that when the packaged product is removed from the treated location, the insect-combating efficacy is lost as regards the package and its contents.

The insect-combating agent in preferred practice of the invention is 2-undecanone or 2-tridecanone, which has been discovered to possess insect-repelling activity, as well as insecticidal activity, for a variety of insect pests to which packaging and packaged products are susceptible to attack. Although described hereinafter with primary reference to 2-undecanone or 2-tridecanone as the insect-combating agent, it will be appreciated that the insect-combating activity evidenced by 2-undecanone or 2-tridecanone with respect to pest species such as ticks, fleas, aphids, cockroaches, flies (horse flies, deer flies, black flies, etc.), gnats, no-see ums, chiggers, thrips, mosquitoes, beetles (e.g., Colorado potato beetle), etc., may be correspondingly achieved with other insect-combating agents and/or with respect to other insect species. It will also be appreciated that the packaging and methods of the invention are applicable to larvae as well as adult insects.

In accordance with the invention, the insect-combating agent is incorporated in the packaging for a packaged product, to provide protection against insect attack to the packaging and/or the packaged product goods. The packaged product goods may be of any suitable type, as for example human and/or veterinary foods/nutritional products, cosmetics, pharmaceuticals, and other organic and inorganic materials. One particularly preferred application of the present invention is veterinary foods/nutritional products and veterinary medicine products that are susceptible to attack by insect pests. These include animal feeds for a wide variety of animals, including dogs, cattle, rabbits, sheep, pigs, horses, birds, and other mammalian, avian and reptilian species, including domesticated as well as wild varieties, and including livestock, pets and service animals.

The invention is described hereinafter with specific reference to 2-undecanone or 2-tridecanone as the illustrative insect-combating agent.

The 2-undecanone or 2-tridecanone insect-combating agent may be incorporated in packaging, as an ingredient of a constituent package structure, e.g., a cellulosic web or sheet material such as cardboard, kraft paper, fiberboard, corrugated paper stock, or the like, which may incorporate the 2-undecanone in the pulped or fiber suspension furnish from which the cellulosic web or sheet material is formed, so that the product web or sheet contains the 2-undecanone or 2-tridecanone as an intrinsic component thereof.

The packaging may be formed of or comprise a polymeric film material, in which the 2-undecanone or 2-tridecanone may be utilized in the manner of a plastics additive, e.g., antioxidants, plasticizers, flame retardants, uv absorbers, etc., being compounded in the melt composition from which the polymeric film is formed, such as by blow molding, rotomolding, extrusion, melt casting, solvent casting, injection molding, etc.

The packaging may be formed of a porous matrix material, such as for example a ceramic, wood, sintered metal, felt, woven fabric, non-woven fabric, or the like, which is impregnated with the 2-undecanone or 2-tridecanone, such as by immersion in a pressure tank containing a 2-undecanone or 2-tridecanone solution, spraying of a solution or suspension of 2-undecanone or 2-tridecanone on the porous matrix material, roller coating of the material, dip coating under ambient pressure conditions, etc.

The insect-combating 2-undecanone or 2-tridecanone component may additionally or alternatively be incorporated in the packaging in a liner, window, insert, bag, or compartment of the package structure, or in a packet, sachet, ampule, or the like, which is deployed in the package as a constituent part thereof.

As a further technique, the 2-undecanone or 2-tridecanone may be formulated in a glue or adhesive bonding medium that is used to assemble or secure the package, such as a glue line at seams of a cardboard box, a bondant medium that is employed to affix a reclosable flap of a container to a top or side portion of the container main body, a sealant medium that is used to provide a moisture-proof seal at a cellophane window joint at a box opening containing a cellophane panel in the window opening for the purpose of viewing the package contents, or other structural application in which a glue, adhesive, bondant and/or sealant is employed to fabricate the finished package. The 2-undecanone or 2-tridecanone may be provided in an insect-combating amount and form, as an ingredient of any structural part, component, or material of the package. 2-undecanone or 2-tridecanone (methyl nonyl ketone) is commercially available from the Sigma-Aldrich Company, P.O. Box 2060, Milwaukee, Wis. 53201 U.S.A. as catalog number U-130-3.

Liquid formulations of 2-undecanone or 2-tridecanone for incorporation in packaging in accordance with the invention, e.g., for spraying, dip coating, impregnation, etc. of package panels or structural components, may be aqueous-based or non-aqueous (i.e., organic solvents), or combinations thereof, and may be employed as foams, gels, suspensions, emulsions, microemulsions or emulsifiable concentrates or the like. The ingredients may include rheological agents, surfactants, emulsifiers, dispersants or polymers.

The concentration of the insect-combating agent can vary widely depending on the nature of the particular formulation, particularly whether it is a concentrate or to be used directly. The insect-combating compound will be present in a composition used to incorporate the compound in packaging, wherein the compound is present in a concentration of at least about 0.0001% by weight, e.g., 10, 50, 99 or 100% by weight of the total composition. A carrier for the compound may be employed and may be from 0.1% to 99.9999% by weight of the total composition. The dry formulations will have from about 0.0001-95% by weight of the pesticide while the liquid formulations will generally have from about 0.0001-60% by weight of the solids in the liquid phase.

The above formulations are those employed for impregnating or coating packaging components, structural parts or materials.

The 2-undecanone or 2-tridecanone insect-combating agent when directly incorporated into a material or structural component of the packaging can be employed in a concentration of from about 0.0001% to about 10% or more by weight of the total packaging material or structure containing same.

In general, the insect-combating 2-undecanone or 2-tridecanone may be employed in any suitable concentration and form that is effective to provide the insect-combating activity desired.

The concentration and form of the 2-undecanone or 2-tridecanone that is usefully employed in a given application of the invention may be readily determined within the skill of the art, based on the disclosure herein, e.g., by varying the concentration and form of the 2-undecanone or 2-tridecanone in a series of packages, against a corresponding control package, and in exposure to corresponding populations of the insect pest(s) sought to be protected against, to assay the results and determine the form and concentration providing a desired level of protection. In such manner, the degree of attack of pests on the package per se and/or the package contents can be determined against a corresponding control lacking the protective incorporation of the 2-undecanone or 2-tridecanone, to establish an effective concentration and form of the insect-combating ingredient.

The form of the insect-combating ingredient may be varied, in that the 2-undecanone or 2-tridecanone may be incorporated in the packaging in a liquid form or as a component of a solid composition, e.g., as an ingredient of a wax coating, lacquer, ink or other material applied to or incorporated in the packaging.

The insect-combating agent of the invention may be used as a single component or as an ingredient in a mixture or combination of two or more insect-combating agents. Further, the insect-combating agent may be deployed in combination with other components or additives in the packaging structure or material, e.g., dispersing agents, dessicants, adjuvants, fillers, etc.

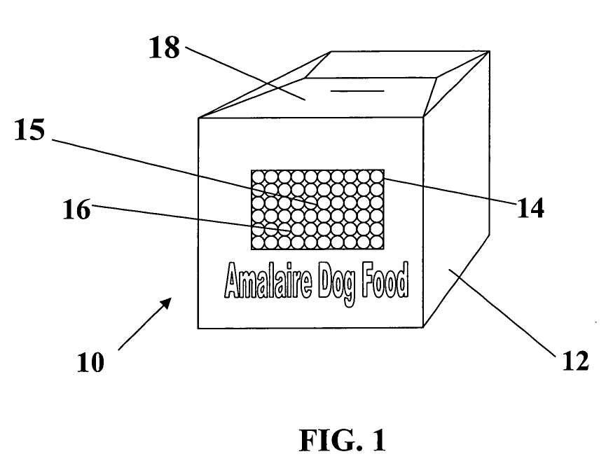

Referring now to the drawing, FIG. 1 is a perspective view of an illustrative packaged product 10 including a box 12 containing a pet food product 16 in a plastic bag 15 visible through window opening 14 of the box 12.

The box 12 includes a reclosable top structure 18 permitting access to the plastic bag 15 and pet food product 16 therein, and reclosable securement of the reclosable top structure 18 of the box, to enable maintenance of freshness, moisture content, etc. of the foodstuff in the box.

In this illustrative packaged product, the insect-combating amount of 2-undecanone or 2-tridecanone may be provided in the cellulosic material of which the box is manufactured, e.g., as an impregnated component that is provided as a component in the furnish liquid from which a cardboard web or sheet material is formed, and subsequently employed to form the body of the box. Alternatively, the 2-undecanone or 2-tridecanone may be provided in the adhesive that is employed to seal the flaps of the reclosable top structure of the box after the sealed product-containing plastic bag is disposed in the box.

Additionally, or alternatively, the 2-undecanone or 2-tridecanone may be provided in an insect-combating amount in the plastic bag material of construction, as for example as a component of the polymeric melt, e.g., polyethylene, polypropylene, polyvinylidenechloride, or other polymeric film material, from which the film material is blown, extruded, molded, solvent cast, or otherwise formed. Similarly, the window opening 14 of the box 12 may include a cellophane or other film material in the opening, in which the 2-undecanone or 2-tridecanone component is contained, or the cellophane or other film material window insert may be adhesively affixed to the inside surface of the box surrounding the window opening, and the 2-undecanone or 2-tridecanone may be provided as a component of such window insert panel adhesive bonding medium.

As a still further expedient, the 2-undecanone or 2-tridecanone may be provided in the interior of the package in an ampule, sachet, or other sub-package structure or component.

It will be appreciated from the foregoing illustrative example that the 2-undecanone or 2-tridecanone component may be deployed in the packaging of the packaged product in any one or more of numerous manners, to provide an insect-combating action protecting the package and its contents from insect attack.

While the foregoing discussion has been primarily directed to applications in which the 2-undecanone or 2-tridecanone is homogeneously or near-homogeneously distributed in the packaging structure, component or material, it will be recognized that the 2-undecanone or 2-tridecanone may be provided in a discontinuous or heterogeneously dispersed manner in the packaging structure, component or material. For example, the sheet or web stock from which the package is formed may be printed with a solution of the 2-undecanone or 2-tridecanone, in which the solution comprises a volatile solvent, e.g., water, alcohol, oil, oil-in-water emulsion or suspension, etc. Such printing may be effected using a patterned roller fed with the solution so that the printed areas contain an insect-combating amount of the 2-undecanone or 2-tridecanone. The printed substrate then may be passed through an oven or elevated temperature exposure zone (e.g., array of heat lamps, etc.) to volatilize and dry the solvent medium, to leave the printed 2-undecanone or 2-tridecanone residue on the substrate surface, present in an insect-combating amount.

Further, while the invention has been described with specific reference to 2-undecanone or 2-tridecanone as an insect-combating agent that is incorporated in packaging, it will be recognized that the utility of the invention is not thus limited, but rather the invention extends to and encompasses the use of other insect-combating agents, including for example, derivatives and analogs of 2-undecanone or 2-tridecanone, other ketones, as well as chemically unrelated entities having insect-combating character.

The insect-combating agent may be present in the packaging in a sustained release form, or in a form that is made available or activated at a specific temperature, humidity level, pH condition, light exposure, etc. It will therefore be appreciated that there are numerous ways in which the insect-combating agent of the invention may be incorporated and deployed in packaging, to provide an insect-combating action to protect the packaging per se, as well as contents of such packaging.

Although the art has contemplated a wide variety of insecticides and insect-repelling agents, the use of insect-combating agents as intrinsic components of packaging, e.g., for containment of human and/or veterinary foodstuffs, cosmetics, pharmaceuticals, and other organic and inorganic materials, has not been envisioned as a practical alternative to the gross application of insect-combating agents to the environment containing packaged products, such as to walls, floor surfaces, shelving, etc. on or near which the packaged products are stored, transported or used. As a consequence, the present invention achieves a substantial advance in the art, obviating the need for such gross applications of insect-combating agents, which by virtue of their localized application are of no effect when the packaged product is removed from the area or environment that has been treated with the insect-combating agent.

While the invention has been described herein with reference to specific features, aspects, and embodiments, it will be recognized that the invention is not thus limited, but is susceptible of implementation in other variations, modifications and embodiments. Accordingly, the invention is intended to be broadly construed to encompass all such other variations, modifications and embodiments, as being within the scope of the invention hereinafter claimed.

1. A packaging article, comprising a package structure, component or material of construction, containing an insect-combating amount of 2-undecanone or 2-tridecanone.

2. The packaging article of claim 1, wherein the package structure, component or material of construction comprises a container structure formed of a sheet or web material having 2-undecanone or 2-tridecanone as an ingredient thereof.

3. The packaging article of claim 1, wherein the package structure, component or material of construction, containing an insect-combating amount of 2-undecanone.

4. The packaging article of claim 1, wherein the package structure, component or material of construction, containing an insect-combating amount of 2-tridecanone.

5. The packaging article of claim 1, wherein the insect-combating ingredient is combatingly effective against insect larvae.

6. The packaging article of claim 1, wherein the insect-combating ingredient is combatingly effective against insect adult species.

7. The packaging article of claim 2, wherein said sheet or web material comprises a cellulosic material.

8. The packaging article of claim 1, wherein the package structure, component or material of construction comprises a polymeric film having 2-undecanone or 2-tridecanone as an ingredient thereof.

9. The packaging article of claim 8, wherein said polymeric film is formed by a method selected from the group consisting of extrusion, blow molding, rotomolding, injection molding, and solvent casting.

10. The packaging article of claim 8, wherein said polymeric film comprises a material selected from the group consisting of polyethylene, polypropylene, polyvinylidene chloride, polyvinylchloride, polysulfone, silicone, and polyacetals.

11. A packaged product comprising a package including a package structure, component or material of construction, containing an insect-combating amount of 2-undecanone or 2-tridecanone, and a contained product in said package.

12. The packaged product of claim 11, wherein the package is susceptible to insect attack.

13. The packaged product of claim 11, wherein the contained product is susceptible to insect attack.

14. The packaged product of claim 11, wherein the package and the contained product are susceptible to insect attack.

15. The packaged product of claim 11, wherein the contained product includes a human food.

16. The packaged product of claim 11, wherein the contained product includes an animal food.

17. The packaged product of claim 16, wherein the animal food comprises a food for an animal selected from the group consisting of dogs, cattle, rabbits, sheep, pigs, horses, and birds.

18. The packaged product of claim 16, wherein the animal food comprises pet food.

19. The packaged product of claim 16, wherein the animal food comprises livestock feed.

20. The packaged product of claim 11, wherein the contained product includes a cosmetic.

21. The packaged product of claim 11, wherein the contained product includes a pharmaceutical.

22. The packaged product of claim 11, wherein the contained product includes a material selected from the group consisting of organic and inorganic materials.

23. A method of combating insects to which a packaging is susceptible, comprising fabricating said packaging with an incorporated insect-combating amount of 2-undecanone or 2-tridecanone.

24. The method of claim 23, wherein said packaging comprises a cellulosic or fabric material in which the 2-undecanone or 2-tridecanone is impregnated.

25. The method of claim 23, wherein said packaging comprises a polymeric film having the 2-undecanone or 2-tridecanone as an additive therein.

26. The method of claim 25, wherein said polymeric film comprises a material selected from the group consisting of polyethylene, polypropylene, polyvinylidene chloride, polyvinylchloride, polysulfone, silicone, and polyacetals.

27. A method of combating insects to which a packaging and/or its contents is susceptible, comprising fabricating said packaging with an incorporated insect-combating amount of 2-undecanone or 2-tridecanone.

28. The method of claim 27, wherein said insects comprise insect larvae.

29. The method of claim 27, wherein said insects comprise adult insects.

30. The method of claim 27, wherein said insects comprise an insect species selected from the group consisting of ticks, fleas, aphids, cockroaches, flies, gnats, no-see ums, chiggers, thrips, mosquitoes, and beetles.

31. The method of claim 27, comprising fabricating said packaging with an incorporated insect-combating amount of 2-undecanone.

32. The method of claim 27, comprising fabricating said packaging with an incorporated insect-combating amount of 2-tridecanone.

33. The method of claim 27, wherein the insect-combating amount of 2-undecanone or 2-tridecanone is incorporated in the packaging as an ingredient of a wax, lacquer, ink or coating applied to or incorporated in the packaging.
